(19) United States
(12) Patent Application Publication
(10) Pub. No.: US 2008/0206194 A1
(43) Pub. Date: Aug. 28, 2008

(54) Method for the treatment of breast cancer

(76) Inventors: Robert I. Glazer, Potomac, MD (US); Yuzhi Yin, Arlington, VA (US); Levy Kopelovich, Annandale, VA (US)

Correspondence Address: M. CARMEN & ASSOCIATES, PLLC, 170 OLD COUNTRY ROAD, SUITE 400, MINEOLA, NY, 11501, US

(21) Appl. No.: 12/069,695

(22) Filed: Feb. 12, 2008

Related U.S. Application Data
(60) Provisional application No. 60/901,901, filed on Feb. 16, 2007.

(51) Int. Cl. A61K 31/4409; A61P 35/00; A61K 31/566; A61K 31/4196; A61K 31/166; A61K 31/4535; A61K 39/395; A61K 48/00; A61K 31/675; A61K 31/704; A61K 31/519; A61K 38/00; A61K 31/7048; A61K 38/19; A61K 38/21; A61K 38/18

(52) U.S. Cl. 424/85.4; 514/353; 514/171; 514/383; 514/619; 514/324; 424/130.1; 424/183.1; 514/44; 514/110; 514/34; 514/262.1; 514/8; 514/27; 424/85.1; 514/12

(57) ABSTRACT

A method for inducing ERα expression in cancer cells in a subject affected with cancer cells which are ERα (−) is disclosed. The method involves administering to the subject an effective amount of a PPARγ antagonist alone or in combination with anti-estrogen therapy.

### PRIORITY

This application claims the benefit under 35 U.S.C. §119 to Provisional Application No. 60/901,901, filed Feb. 16, 2007 and entitled “METHOD FOR THE TREATMENT OF BREAST CANCER”, the contents of which are incorporated by reference herein.

### BACKGROUND OF THE INVENTION

1. Technical Field

The present invention generally relates to a method for inhibiting the peroxisome proliferator-activated receptor gamma (PPARγ) to induce estrogen receptor alpha (ERα) expression, wherein ERα becomes a target that is modulated by specific inhibitors causing a reduction in cancer growth.

2. Description of the Related Art

The peroxisome proliferator activator receptors (“PPARs”) are members of the nuclear receptor superfamily, which are ligand-activated transcription factors regulating gene expression. Various subtypes of PPARs have been discovered. These include PPARα, PPARγ and PPARδ. In the presence of PPAR ligands, the PPAR family regulates the transcription of targeted genes. The PPAR receptors were originally identified as regulators of adipocyte differentiation and lipid metabolism. Recently, PPARγ has been shown to be expressed in cells of the immune system including both T cells and macrophage. The endogenous ligands for PPARγ are thought to be lipids, although there are also a number of synthetic drugs (e.g., thiazolidinediones rosiglitazone, ciglitazone and pioglitazone) that have been shown to regulate lipid and sugar metabolism via PPARγ.

The role of PPARγ as a regulator of the immune system is receiving a great deal of attention. U.S. Patent Application Publication No. 20040122059 discloses that PPARγ was observed to be highly expressed in myeloid cells and activated by endogenous ligands such as 15-deoxy-Delta(12,14)-prostaglandin J(2) (PGJ2) as well as synthetic ligands that regulate macrophage activation. The use of PPARγ antagonists to treat ocular inflammation is also known. See, e.g., Leesnitzer et al., Functional Consequences of Cysteine Modification in the Ligand Binding Sites of Peroxisome Proliferator Activated Receptors by GW9662, Biochemistry 41, pp. 6640-6650, (2002).

U.S. Pat. No. 6,316,465 (“the '465 patent”) discloses methods of treating diseases of ocular tissues expressing the nuclear receptor PPARγ, by inhibiting the inflammatory response, the neovascularization and angiogenesis, and programmed cell death (apoptosis) in these target tissues. The method involves administering to a human or animal in need of treatment an effective amount of a compound that modifies the activity of PPARγ, or pharmaceutically acceptable salts and solvates thereof. The '465 patent further discloses novel compounds and methods for their synthesis.

The use of RAR antagonists as hormone modulators is also known. U.S. Pat. No. 6,436,993 discloses that retinoic acid receptor (RAR) antagonists are capable of modulating processes mediated by other members of the steroid/thyroid hormone receptor superfamily, including permissive receptors such as PPARs (e.g., PPARα, PPARγ and PPARδ). It has been discovered that RAR antagonists, in combination with agonists for members of the steroid/thyroid hormone receptor superfamily, are capable of inducing and/or enhancing processes mediated by such members.

Breast cancer is the second most common cancer in women. Although breast cancer tumor cells predominantly express ERα positive (+), 20 to 30% of breast cancer tumors do not express ERα negative (−) and, therefore, are not amenable to anti-estrogen therapy (see, e.g., Moy et al., Estrogen receptor pathway: resistance to endocrine therapy and new therapeutic approaches, Clin. Cancer Res., 12, pp. 4790-93, (2006)). In addition, a high proportion of ERα (−) breast cancer is particularly evident among African-American women, descendents of African women elsewhere and women in major areas of the African continent. The overall cure rate for breast cancer is directly related to the stage of the disease and the type of treatment used. However, while survival is increased in patients having tumor cells which express ERα (+), survival outcome in ERα (−) tumor patients is poor.

Accordingly, it would be desirable to provide improved methods for the treatment of cancer such as breast cancer.

### SUMMARY OF THE INVENTION

In accordance with one embodiment of the present invention, a method for inducing ERα expression in cancer cells of a subject affected with cancer cells which are ERα (−) is provided comprising administering to the subject an effective amount of a PPARγ antagonist.

In accordance with a second embodiment of the present invention, a method of treating a subject affected with cancer cells which are ERα (−) is provided comprising administering to the subject an effective amount of a PPARγ antagonist capable of inducing ERα expression in the cancer cells and administering an effective amount of an anti-estrogen agent.

In accordance with a third embodiment of the present invention, a composition is provided comprising (a) a PPARγ antagonist and (b) an anti-estrogen agent.

By administering a PPARγ antagonist to a subject affected with cancer cells which are ERα (−), it is believed that a sufficient amount of the cancer cells can become ERα (+) to allow for anti-estrogen therapy to treat the cancer cells. In other words, by inducing the expression of ERα to a sufficient level, the tumor growth can become dependent on ERα (+) and therefore responsive to anti-estrogen therapy.

The term “treatment” as used throughout the specification means: (1) preventing such disease from occurring in a subject who may be predisposed to these diseases but who has not yet been diagnosed as having them; (2) inhibiting these diseases, i.e., arresting or slowing down their development; or (3) ameliorating or relieving the symptoms of these diseases.

The term “effective amount” as used throughout the specification means an amount of a compound necessary to obtain a detectable clinical effect. The detectable effect may include, for example and without limitation, inducing ERα expression or can be a therapeutic effect such as inhibiting the growth of undesired tissue or malignant cells, inhibition of tumor cell growth, decreased levels of an estrogen receptor transcript or protein or both. The precise effective amount for a subject will depend upon the subject's size and health, the nature and severity of the condition to be treated, and the like. The effective amount for a given situation can be determined by routine experimentation based on the information provided herein.

The term “subject” or “a patient” or “a host” as used herein refers to mammalian animals, preferably human.

### BRIEF DESCRIPTION OF THE DRAWINGS

FIG. 1 illustrates ER detection by immunochemical staining with a rabbit polyclonal antibody of dimethylbenz(α)anthracene (DMBA)/progestin-induced mammary carcinomas of wild-type FVB mice, transgenic MMTV-Pax8PPARγ FVB mice and GW9662-treated mice, with 40 mg/kg, subcutaneously, weekly, for 12 weeks. MMTV-Pax8PPARγ FVB mice and GW9662-treated wild-type mice, but not wild-type mice treated with vehicle alone, express substantial ERα staining.

FIG. 2 is a bar graph showing the total number of mammary tumors appearing in transgenic MMTV-Pax8PPARγ FVB mice treated with either vehicle or ERα antagonist fulvestrant, 200 mg/kg subcutaneously once per week for three months after carcinogen administration.

### DESCRIPTION OF PREFERRED EMBODIMENTS

One embodiment of the present invention is directed to a method for inducing ERα expression in an ERα (−) cancer cell of a subject, thereby making the cell responsive to anti-estrogen therapy. By administering the PPARγ antagonist to the subject, ERα expression will be induced such that the subject will be able to receive treatment for cancer that is now responsive to anti-estrogen treatment. In a preferred embodiment, the estrogen receptor is ERα. Individuals who can be treated with the methods of the present invention include those affected with ERα associated cancers including osteosarcomas, pituitary adenomas, testicular, uterine, ovarian and breast cancers. Different types of breast cancers include, but are not limited to, ductal carcinoma in situ (DCIS), infiltrating (or invasive) ductal carcinoma (IDC), or infiltrating (or invasive) lobular carcinoma (ILC). In one preferred embodiment, the individual is affected with breast cancer wherein the cancer cells are ERα (−).

Generally, the method involves administering to the subject an effective amount of a PPARγ antagonist capable of inducing ERα expression. Representative examples of a suitable PPARγ antagonist that may be used herein include those compounds having the formula:

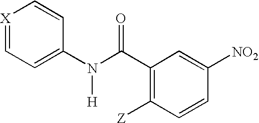

where X can be a CH or N, Z is Cl, F, Br or I, or a pharmaceutically acceptable salt or derivative thereof. In one embodiment, X may be a CH and Z is Cl. In accordance with this embodiment, the PPAR-gamma antagonist is GW9662. In another embodiment, X may be a N and Z is Cl. In accordance with this embodiment, the PPARγ antagonist is T0070907. GW9662 and T0070907 are known in the art, e.g., H. Lee et al., Nuclear Medicine and Biology, Synthesis and evaluation of a bromine-76-labeled PPARγ antagonist 2-bromo-5-nitro-N-phenylbenzamide, Vol. 33, pp. 847-854 (2006), the contents of which are incorporated by reference herein. GW9662 and T0070907 are also commercially available from, for example, Sigma-Aldrich (St. Louis, Mo.) and Cayman Chemical Co. (Ann Arbor, Mich.).

Once ERα expression has been induced, an effective amount of one or more anti-estrogen agents can be administered. In one embodiment, the anti-estrogen agents are administered following administration to the subject of the effective amount of a PPARγ antagonist. In another embodiment, the PPARγ antagonist and anti-estrogen agents are administered simultaneously. In this embodiment, it may be necessary to administer the anti-estrogen agents in a sustained release manner. Suitable anti-estrogen agents for use herein include, but are not limited to, ERα antagonists, aromatase inhibitors, selective ERα modulators (SERMs) and the like and mixtures thereof. Suitable ERα antagonists include, but are not limited to, fulvestrant and the like and mixtures thereof. Suitable aromatase inhibitors include, but are not limited to, letrozole and the like and mixtures thereof. Suitable SERMs include, but are not limited to, tamoxifen, raloxifene and the like and mixtures thereof.

If desired, one or more anti-cancer drugs may be administered to the subject with the agents described herein together or in any order, i.e., before, during or after administration of the PPARγ antagonist and/or anti-estrogen agents. In one embodiment, the anti-cancer drug used in combination with one or more PPARγ antagonist and anti-estrogen agents described herein include, but are not limited to, an antibody, an immunoconjugate, antibody-immunomodulator fusion protein, an antibody-toxin fusion protein, a cytotoxic agent, a serine/threonine kinase inhibitor, a tyrosine kinase inhibitor, a proteasome inhibitor, a thalidomide analog, a histone deacetylase inhibitor, a cyclooxygenase inhibitor, a hormone, a hormone antagonist, an antisense oligonucleotide, an interference RNA, and an immunomodulator.

In one embodiment, the anti-cancer drug used in combination with one or more PPARγ antagonist and/or anti-estrogen agents described herein include, but are not limited to, cyclophosphamide, etoposide, vincristine, procarbazine, carmustine, doxorubicin, methotrexate, bleomycin, and dexamethasone.

In another embodiment of the present invention, the anti-cancer drugs used in combination with one or more PPARγ antagonist and anti-estrogen agents described herein include, but are not limited to, interferons (e.g., IFN-γ gamma, beta and/or alpha), lymphokines, cytokines (e.g., interleukin-2 (IL-2), IL-18, IL-11), growth factors (e.g., platelet derived growth factor (PDGF), tumor necrosis factor (TNF) and epidermal growth factor (EGF)) and the like and mixtures thereof.

In one embodiment, the additional agents or factors suitable for use in a combined therapy may be any chemical compound or treatment method that induces DNA damage when applied to a cell. Such agents and factors include radiation and waves that induce DNA damage such as gamma-irradiation, X-rays, UV-irradiation, microwaves, electronic emissions, and the like and mixtures thereof. A variety of chemical compounds, also described as “chemotherapeutic agents,” function to induce DNA damage, all of which are intended to be of use in the combined treatment methods disclosed herein. Chemotherapeutic agents contemplated for use herein include, but are not limited to, adriamycin, 5-fluorouracil (5FU), etoposide (VP-16), camptothecin, actinomycin-D, mitomycin C, cisplatin (CDDP) and the like and mixtures thereof. It is also contemplated herein that the use of a combination of one or more DNA damaging agents may be required depending on the subject and the condition of the subject, whether radiation-based or actual compounds, such as the use of X-rays with cisplatin or the use of cisplatin with etoposide.

The compounds employed in the methods of the present invention may be formulated with one or more pharmaceutically acceptable ingredients in accordance with known and established practice. Thus, the compounds can be formulated as a liquid, powder, elixir, injectable solution, etc. Formulations for oral use can be provided as tablets or hard capsules wherein the pharmacologically active ingredients are mixed with an inert solid diluent such as calcium carbonate, calcium phosphate or kaolin, or as soft gelatin capsules wherein the active ingredients are mixed with water or miscible solvents such as propylene glycol; PEG's and ethanol, or an oleaginous medium, e.g., peanut oil, liquid paraffin or olive oil.

For oral therapeutic administration, the active compound may be incorporated with an excipient and used in the form of, for example, ingestible tablets, buccal tablets, troches, capsules, elixirs, suspensions, syrups, wafers, and the like. Such compositions and preparations should contain at least about 0.1% of the antagonist. The percentage of the compositions and preparations may be varied such that a suitable dosage will be obtained. Preferred compositions or preparations according to the present invention are prepared so that an oral dosage unit form contains between about 1 and 500 mg of active compound, although other dosage forms may be used. Suitable pharmaceutical compositions of the antagonists are known in the art.

For topical administration to the epidermis the compounds can be formulated as creams, gels, ointments or lotions or as transdermal patches. Such compounds can, for example, be formulated with an aqueous or oily base with the addition of suitable thickening, gelling, emulsifying, stabilizing, dispersing, suspending, and/or coloring agents.

The compounds can also be formulated as depot preparations. Such long acting formulations can be administered by implantation (for example, subcutaneously or intramuscularly) or by intramuscular injection. A pharmaceutical form suitable as an injectable includes sterile aqueous solutions or dispersions and sterile powders for the extemporaneous preparation of sterile injectable solutions or dispersions. Thus, for example, the compounds can be formulated with suitable polymeric or hydrophobic materials (for example, an emulsion in an acceptable oil) or ion exchange resins, or as sparingly soluble derivatives, for example, as a sparingly soluble salt. In all cases, the form must be sterile and must be fluid to the extent that easy syringability exists. It may be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like) and suitable mixtures thereof. The proper fluidity can be maintained, for example, by the use of a coating such as lecithin, by the maintenance of the required particle size in the case of dispersion and by the use or surfactants. The prevention of the action of microorganisms can be brought about by various antibacterial and antifungal agents, for example, parabens, chlorobutanol, phenol, sorbic acid, thimerosal, and the like.

The compounds can be formulated for parenteral administration by injection, conveniently intravenous, intramuscular or subcutaneous injection, for example by bolus injection or continuous intravenous infusion. Formulations for injection can be presented in unit dosage from, e.g., in ampoules or in multi-dose containers, with an added preservative. The compounds can take such forms as suspensions, solutions or emulsions in oily or aqueous vehicles, and can contain formulatory agents such as suspending, stabilizing and/or dispersing agents. Alternatively, the compounds can be in powder form for constitution with a suitable vehicle, e.g. sterile pyrogen-free water, before use.

The compounds can also be formulated in rectal compositions such as suppositories or retention enemas. e.g., containing conventional suppository bases such as cocoa butter or other glyceride.

For intranasal administration, the compounds can be used, for example, as a liquid spray, as a powder or in the form of drops.

For administration by inhalation, the compounds are conveniently delivered in the form of an aerosol spray presentation from pressurized packs or a nebulizer, with the use of a suitable propellant, e.g. dichlorodifluoromethane, trichlorofluoromethane, dichlorotetrafluoroethane, tetrafluoroethane, heptafluoropropane, carbon dioxide or other suitable gas. In the case of a pressurized aerosol the dosage unit can be determined by providing a valve to deliver a metered amount. Capsules and cartridges of, for example, gelatin, for use in an inhaler or insulator can be formulated containing a powder mix of a compound of the invention and a suitable powder base such as lactose or starch.

Aqueous suspensions can include pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. The aqueous suspensions can also contain one or more preservatives, e.g., ethyl-or-n-propyl-p-hydroxy benzoate, one or more coloring agents, one or more flavoring agents and one or more sweetening agents, such as sucrose, saccharin or sodium or calcium cyclamate.

The antagonists and compositions containing at least the antagonists may be administered as frequently as necessary in order to obtain the desired expression level of ER and treatment of cancer. In accordance with this embodiment, the antagonists and composition may be administered more than once a day, daily, every other day, 2 times per week, once a month, 2 times a month, etc.

The following examples are provided to enable one skilled in the art to practice the invention and are merely illustrative of the invention. The examples should not be read as limiting the scope of the invention as defined in the claims.

### EXAMPLE 1

Synthesis of GW9662 (2-Chloro-5-nitrobenzanilide).

To a stirred solution of 2-chloro-5-nitrobenzoyl chloride (5.03 g, 22.9 mmol) and triethylamine (3.51 mL, 25.1 mmol) in CH₂Cl₂ maintained under nitrogen at 0° C. was added dropwise aniline (2.19 mL, 24.0 mmol). The resulting solution was stirred for 5 min at 0° C. and then for 15 min at room temperature. This solution was then diluted with ethyl acetate (EtOAc) (300 mL) and washed sequentially with 1.0 M HCl, water, 1.0 M NaHCO₃, and brine (100 mL each). The organic solution was then dried over MgSO₄ and concentrated by rotary evaporation to give a light yellow solid (5.32 g) which was recrystallized from EtOAc to provide the title compound as a white solid (3.34 g, 53%): mp 155-156° C.; 1H NMR (CDCl3, 400 MHz) % 8.63 (d, 1H, J) 2.7), 8.28 (dd, 1H, J) 2.7, 8.9), 7.81 (br s, 1H) 7.68-7.63 (m, 3H), 7.42 (t, 2H, J) 7.9), 7.23 (t, 1H, J) 7.5); MS (ES−) mle 275.1 (MH)−; Anal. Calcd. for C13H9C11N2O3: C, 56.43; H, 3.28; N, 10.13; Found: C, 56.33; H, 3.30; N, 10.03.

### EXAMPLE 2

Mammary carcinogenesis was induced in female wild-type FVB/N or MMTV-Pax8PPARγ transgenic mice purchased from Charles River Laboratories (Wilmington, Mass.) by subcutaneous injection of 600 mg/kg medroxyprogesterone acetate suspension (150 mg/ml, Sicor Pharmaceuticals Inc., Irvine, Calif.), followed one week later by four weekly oral doses of 1 mg dimethylbenz(a)anthracene (DMBA) dissolved in cottonseed oil (10 mg/ml). Following the last dose of DMBA, mice were injected subcutaneously once a week with 40 mg/kg GW9662 dissolved in cottonseed oil (10 mg/ml). GW9662 was synthesized according to Leesnitzer et al., Functional Consequences of Cysteine Modification in the Ligand Binding Sites of Peroxisome Proliferator Activated Receptors by GW9662, Biochemistry 41, pp. 6640-6650, (2002) and provided under a contract with the National Cancer Institute, NIH, Bethesda, Md. The body weight of the mice was in the range of 20 to 25 g at the day of treatment initiation. The mice were healthy, not previously used in other experimental procedures. Tumors in wild-type mice were ERα(−) and tumors in MMTV-Pax8PPARγ transgenic mice and GW9662-treated mice were ERα(+).

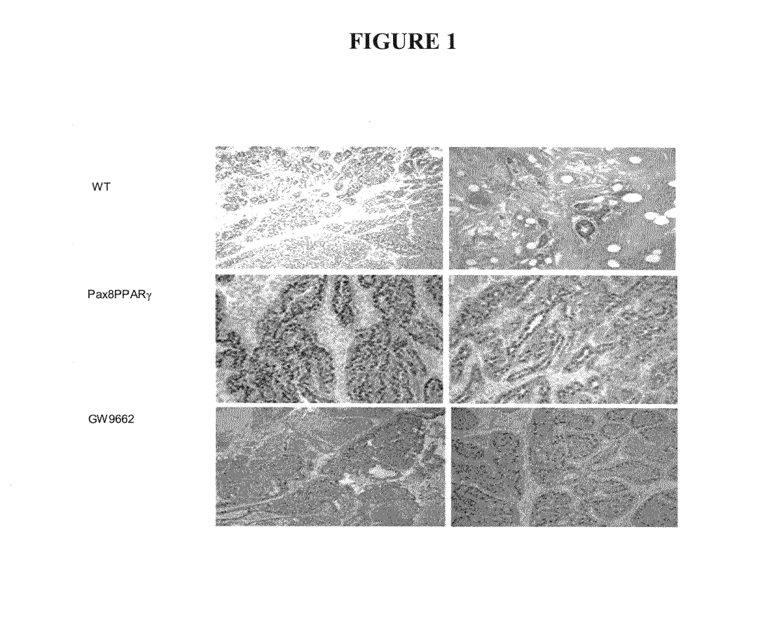

Mammary carcinomas from transgenic MMTV-Pax8PPARγ FVB mice stained intensely for ERα, particularly in the basal layer of cells, as compared to low ERα staining in ductal epithelium from wild-type FVB mice (see FIG. 1). Mammary carcinomas from GW9662-treated mice express a pattern of ERα expression similar to MMTV-Pax8PPARγ mice (Magnification 400×) (see FIG. 1). In this instance, GW9662 mimicked Pax8PPARγ, a dominant-negative PPARγ transgene expressed in transgenic mice, by inducing ERα expression, and demonstrated a pharmacological effect equivalent to complete suppression of endogenous PPARγ activity. ERα was detected by immunochemical staining with a rabbit polyclonal antibody (Santa Cruz Biotechnology, sc-542) diluted 1:1000.

### EXAMPLE 3

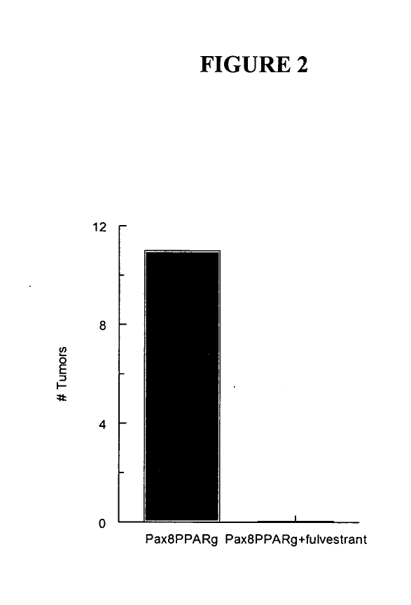

Mammary carcinogenesis (see, e.g., Yin et al., Characterization of medroxyprogesterone and DMBA-induced multilineage mammary tumors by gene expression. Mol. Carcinogenesis 42:pp. 42-50, (2005)) was induced in Pax8PPARγ transgenic mice, which mammary carcinomas are ER(+) and were treated once a week for three months with the ER antagonist fulvestrant at a dose of 200 mg/kg administered subcutaneously in an oil emulsion. It was seen that fulvestrant completely inhibited tumor formation in the Pax8PPARγ mice following carcinogenesis. Each experimental group consisted of 6 mice. FIG. 2 indicates the total number of mammary tumors appearing three months after carcinogen administration.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto

1. A method for inducing estrogen receptor alpha (ERα) expression in cancer cells of a subject affected with cancer cells which are ERα (−), the method comprising administering to the subject an effective amount of a peroxisome proliferator-activated receptor gamma (PPARγ) antagonist.

2. The method of claim 1, wherein the PPARγ antagonist is of the formula: 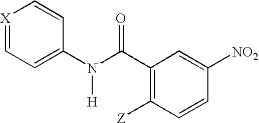
where X is CH or N and Z is Cl, F, Br or I, or a pharmaceutically acceptable salt or derivative thereof.

3. The method of claim 2, wherein Z is Cl.

4. The method of claim 1, wherein the PPARγ antagonist is GW9662 or T0070907.

5. The method of claim 1, further comprising the step of administering to the subject an effective amount of an anti-estrogen agent.

6. The method of claim 5, wherein the anti-estrogen agent is selected from the group consisting of an ERα antagonist, aromatase inhibitor, selective estrogen receptor modulator (SERM) and mixtures thereof.

7. The method of claim 6, wherein the anti-estrogen agent is selected from the group consisting of fulvestrant, letrozole, tamoxifen, raloxifene and mixtures thereof.

8. The method of claim 5, further comprising the step of administering to the subject an anti-cancer agent.

9. The method of claim 8, wherein the anti-cancer agent is selected from the group consisting of an antibody, an immunoconjugate, an antibody-immunomodulator fusion protein, an antibody-toxin fusion protein, a cytotoxic agent, a serine/threonine kinase inhibitor, a tyrosine kinase inhibitor, a proteasome inhibitor, a thalidomide analog, a histone deacetylase inhibitor, a cyclooxygenase inhibitor, a hormone, a hormone antagonist, an antisense oligonucleotide, an interference RNA, an immunomodulator and mixtures thereof.

10. The method of claim 8, wherein the anti-cancer agent is selected from the group consisting of cyclophosphamide, etoposide, vincristine, procarbazine, carmustine, doxorubicin, methotrexate, bleomycin, dexamethasone and mixtures thereof.

11. The method of claim 8, wherein the anti-cancer agent is an immunomodulator selected from the group consisting of an interferon, lymphokine, cytokine, growth factor and mixtures thereof.

12. A method of treating a subject affected with cancer cells which are ERα (−), the method comprising administering to the subject an effective amount of a PPARγ antagonist capable of inducing ERα expression in the cancer cells and administering an effective amount of an anti-estrogen agent.

13. The method of claim 12, wherein the cancer cells are breast cancer cells.

14. A composition comprising (a) a PPARγ antagonist and (b) an anti-estrogen agent.

15. The composition of claim 14, wherein the PPARγ antagonist is of the formula: 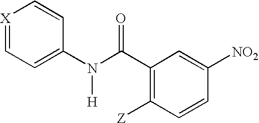
where X is CH or N and Z is Cl, F, Br or I, or a pharmaceutically acceptable salt or derivative thereof.

16. The composition of claim 15, wherein X is CH and Z is Cl.

17. The composition of claim 14, wherein the PPARγ antagonist is GW9662 or T0070907.

18. The composition of claim 15, wherein X is N and Z is Cl.

19. The composition of claim 14, wherein the anti-estrogen agent is selected from the group consisting of an ERα antagonist, aromatase inhibitor, SERM and mixtures thereof.

20. The composition of claim 14, wherein the anti-estrogen agent is selected from the group consisting of fulvestrant, letrozole, tamoxifen, raloxifene and mixtures thereof.
